(19) United States
(12) Patent Application Publication
(10) Pub. No.: US 2002/0072698 A1
(43) Pub. Date: Jun. 13, 2002

(54) Pumping device for helping severe failing of left ventricle or heart

(76) Inventors: Jih Chung Chiang, Taichung (TW); Chin Sheng Tung, Tainan (TW); Kevin Kuo Tsai Cheng, Tainan (TW); Shih Hsiung Chen, Tainan (TW); Tzu Min Kao, Yun Lin Hsien (TW); Jeng Wei, Taipei (TW); Pong Jeu Lu, Tainan (TW)

Correspondence Address: CHARLES E. BAXLEY, 5th Floor, 59 John Street, New York, NY, 10038, US

(21) Appl. No.: 10/045,379

(22) Filed: Jan. 15, 2002

(30) Foreign Application Priority Data
Dec. 13, 2000 (TW) 089126683

(51) Int. Cl. A61M 37/00; A61M 5/00; A61M 1/10

(52) U.S. Cl. 604/6.11; 623/3.16; 623/3.26; 604/9

(57) ABSTRACT

A blood pumping device includes a receptacle for receiving blood and having a non-valved inlet and outlet openings coupled to an aorta and the femoral artery. The receptacle includes a surface having a peripheral portion coupled to a peripheral portion of a diaphragm, and having a port coupled to a pump. The pump may shrink the diaphragm to draw the blood into the receptacle and may expand the diaphragm to pump the blood in the chamber of the receptacle out of the inlet opening and the outlet opening of the receptacle and into the aorta and the femoral artery of the patient.

### BACKGROUND OF THE INVENTION

1. Field of the Invention

The present invention relates to a pumping device, and more particularly to a blood pumping device for draining the blood out of the left ventricle, and for helping recovery of the heart, or for facilitating the blood circulation of the patients.

2. Description of the Prior Art

As shown in FIGS. 6 and 7, illustrated is a typical blood pumping device for pumping the blood out of the left ventricle and for helping or facilitating the blood circulation of the patients. The blood pumping device includes a receptacle 1 having a chamber 10 formed or provided therein, and having an inlet 12 for coupling to the heart, such as the left ventricle of the heart for receiving the blood from the heart and for allowing the blood to flow into the chamber 10 of the receptacle, and having an outlet 11 for coupling to the artery, such as the aorta of the patient and for allowing the blood to be pumped to flow to the aorta, in order to facilitate the blood circulation. Two check valves 110, 120 are disposed in the outlet 11 and the inlet 12 of the receptacle 1 respectively for preventing the blood from flowing backward. A resilient diaphragm 130 is formed or provided in the receptacle 1 and attached to or extended from the flat bottom surface 13 of the receptacle 1. A tube 14 is formed or provided or attached between the diaphragm 130 and the receptacle 1. A hose 2 may be engaged through the bore 140 of the tube 14 and coupled to the space formed by the diaphragm 130 for filling or drawing or pumping an air or a fluid to shrink or to expand the diaphragm 130, and for drawing the blood into the chamber 10 of the receptacle 1 and for pumping the blood in the chamber 10 of the receptacle 1 out to the aorta of the patient.

However, the heart of the patient is required to be punctuated for attaching or for coupling the typical blood pumping device. Furthermore, the typical blood pumping device includes many dead angles or dead portions that the blood may not be pumped out of the receptacle 1. Particularly, the portions around or near the inlet 12 and the outlet 11 of the receptacle 1, and the portions 3 around the tube 14, such that the thrombosis problems may be occurred, and such that the typical blood pumping device is required to be removed from the patient and required to be cleaned or to be replaced with the other ones. In additional, the check valves 110, 120 of the blood pumping device may be easily become fail or may be easily disordered.

The present invention has arisen to mitigate and/or obviate the afore-described disadvantages of the conventional blood pumping devices.

### SUMMARY OF THE INVENTION

The primary objective of the present invention is to provide a blood pumping device for draining the blood out of the left ventricle and for helping recovery of the heart or for facilitating the blood circulation of the patients.

In accordance with one aspect of the invention, there is provided a blood pumping device comprising a receptacle including a chamber formed therein for receiving blood, and including a non-valved inlet or larger opening for coupling to an aorta of a patient and for receiving blood from the aorta, and including a non-valved out let or smaller opening for outward flowing of the blood to the femoral artery. The receptacle includes a surface having a peripheral portion and having a port provided therein, a resilient diaphragm is received in the chamber of the receptacle and includes a peripheral portion coupled to the peripheral portion of the surface of the receptacle. The diaphragm may be expanded to pump the blood into the chamber of the receptacle and to drain the blood in the chamber of the receptacle out of the inlet or larger opening and the outlet or smaller opening of the receptacle.

The pumping means includes a pump coupled to the port of the receptacle for deflating the diaphragm to draw the blood into the chamber of the receptacle, and for expanding the diaphragm to force and to pump the blood in the chamber of the receptacle out of the inlet opening and the outlet opening of the receptacle.

Further objectives and advantages of the present invention will become apparent from a careful reading of a detailed description provided hereinbelow, with appropriate reference to accompanying drawings.

### BRIEF DESCRIPTION OF THE DRAWINGS

FIG. 1 is a perspective view of a blood pumping device in accordance with the present invention;

FIG. 2 is a cross sectional view taken along lines 2-2 of FIG. 1;

FIG. 3 is a schematic view illustrating the engagement or the attachment of the blood pumping device in the human body;

FIG. 4 is a partial cross sectional view illustrating the engagement or the attachment of the blood pumping device to the aorta of the human body;

FIG. 5 is a partial cross sectional view similar to FIG. 4, illustrating the operation of the blood pumping device or the operation of the diaphragm;

FIG. 6 is a perspective view showing a typical blood pumping device; and

FIG. 7 is a cross sectional view taken along lines 7-7 of FIG. 6, for showing the inner structure of the typical blood pumping device.

### DETAILED DESCRIPTION OF THE PREFERRED EMBODIMENT

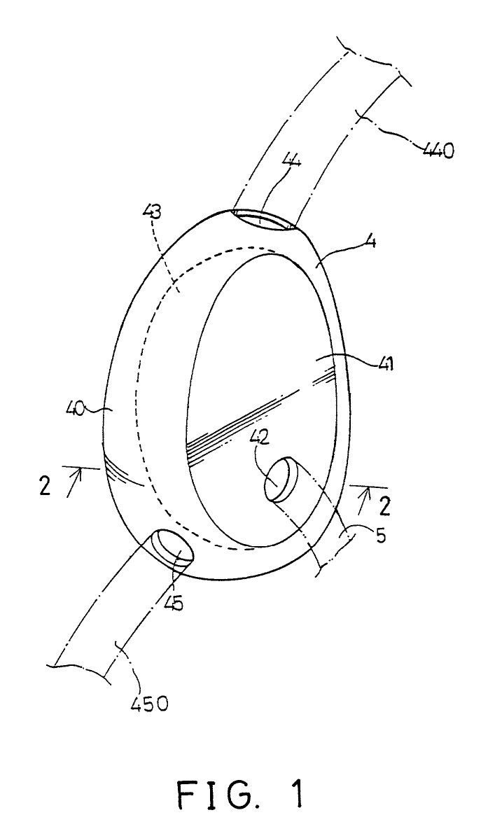

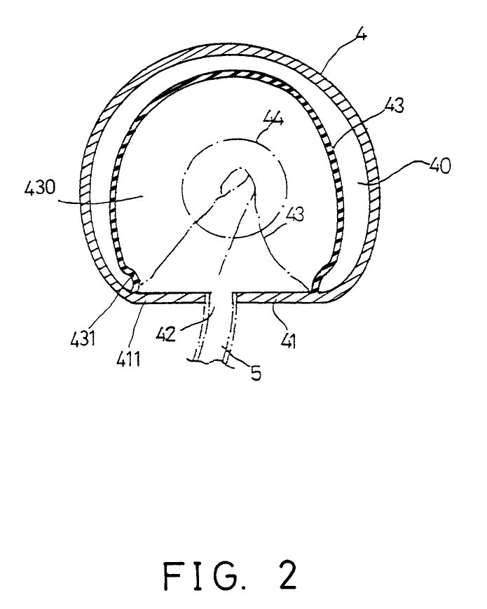

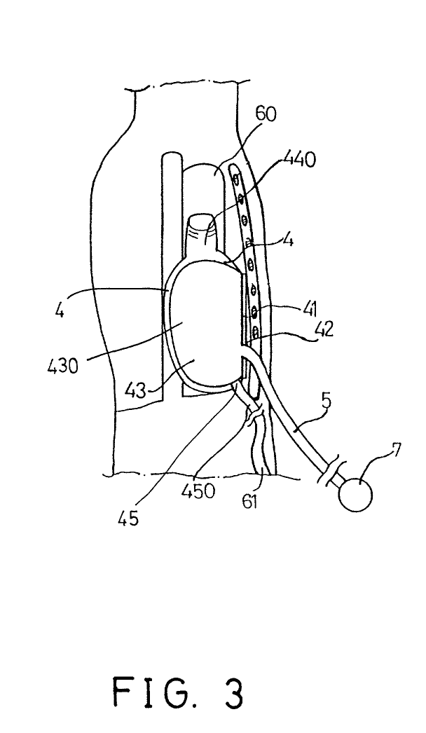

Referring to the drawings, and initially to FIGS. 1-4, a blood pumping device in accordance with the present invention comprises a receptacle 4 including a chamber 40 formed or provided therein for receiving blood, and including an inlet or a larger opening 44 for coupling to the ascending aorta and for receiving the blood from the ascending aorta and for allowing the blood to flow into the chamber 40 of the receptacle. For example, as shown in FIGS. 3-5, a hose 440 may be used to couple the inlet or the larger opening 44 of the receptacle 4 to the aorta 60, such as the portion of the aorta 60 that is located between the heart 6 and the aortic arch 63, and particularly between the heart 6 and the innominate artery 64 that is coupled to the aortic arch 63.

The receptacle 4 includes an outlet or a smaller opening 45 for coupling to the artery, such as the femoral artery 61 of the patient and for allowing the blood to be pumped to flow to the femoral artery 61, in order to facilitate the blood circulation. It is to be noted that no check valves are required to be provided or disposed in the outlet or the smaller opening 45 and the inlet or the larger opening 44 of the receptacle 4. A check valve 8 is required to be disposed in the aorta 60 for preventing the blood from flowing backward from the aortic arch 63 to the aorta 60, and for allowing the blood to be pumped to flow into the aortic arch 63 only. The common femoral artery may be snared to decrease blood from flowing backward through the outlet or the smaller opening 45 to the receptacle 4.

The receptacle 4 includes a flat peripheral surface or a flat bottom or side surface 41 having a port 42 formed or provided therein. A capsule or an envelope or a rubber or resilient diaphragm 43 is formed or provided in the receptacle 4 and attached to or extended from the flat surface 41 of the receptacle 4. The diaphragm 43 includes a space 430 formed or provided therein, and includes a peripheral portion 431 coupled to or secured to the peripheral portion 411 of the surface 41, for example. A pump, such as a hand pump 7 or the like (FIG. 3) is coupled to the port 42 of the receptacle 4 with a hose 5, for filling or drawing or pumping an air or a fluid into or out of the space 430 formed or surrounded by the diaphragm 43, and for drawing the blood into the chamber 40 of the receptacle 4 from the aorta 60, and for pumping the blood in the chamber 40 of the receptacle 4 out to the aorta and femoral artery 61 of the patient.

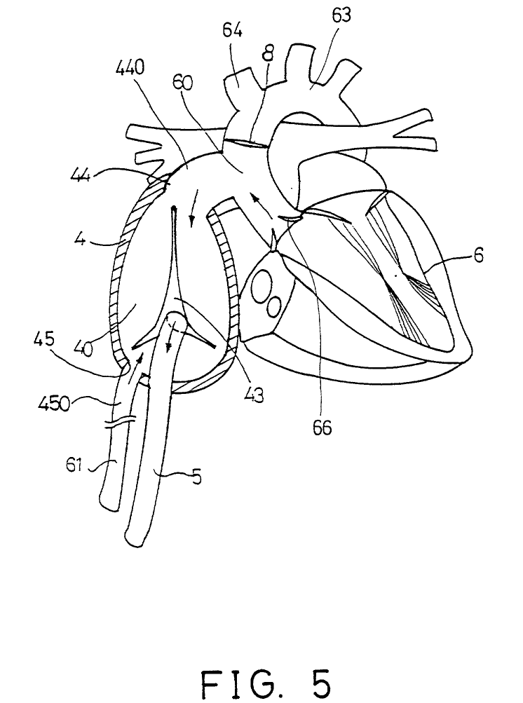

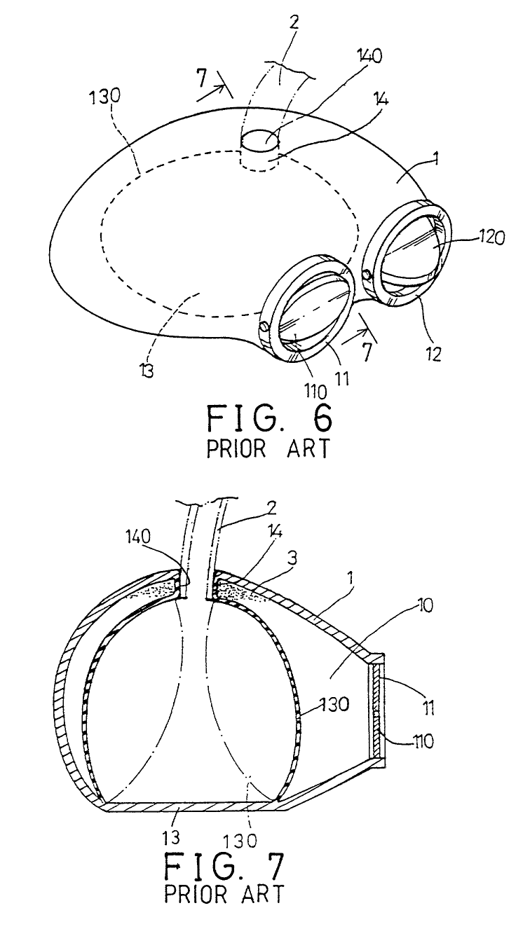

In operation, as shown in FIG. 3, the hand or other pumps 7 may draw or pump the air or the fluid to deflate the diaphragm 43, or to expand the diaphragm 43 (FIG. 5), such that the blood may be drawn from the aorta 60 into the chamber 40 of the receptacle 4. The check valve 8 in the aorta 60 may prevent the blood from flowing backward from the aortic arch 63 to the aorta 60, and the aortic semilunar valve 66 may be used to prevent the blood from flowing backward from the aorta 60 to the heart 6 and allows the blood to flow out of the left ventricle of the heart 6 only. The common femoral artery 61 may be snared during operation to decrease the amount of blood from backward flowing to the receptacle 4, such that the blood may be effectively drawn from the left ventricle of the heart 6 into the receptacle 4 when the diaphragm 43 is deflated.

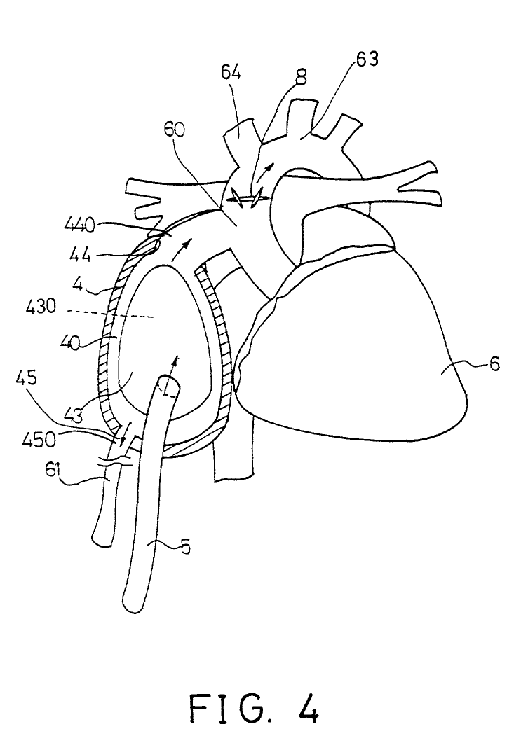

As shown in FIG. 4, when the diaphragm 43 is inflated by the hand or the other pumps 7 which may force or pump the air or the fluid, through the hose 5, into the space 430 enveloped by the diaphragm 43, to expand the diaphragm 43. The blood in the chamber 40 of the receptacle 4 may thus be forced to flow out of the receptacle 4 and to flow into the aortic arch 63 and the femoral artery 61. The aortic semilunar valve 66 may prevent the blood from flowing backward from the aorta 60 into the left ventricle of the heart 6 at this moment, and the check valve 8 in the aorta 60 allows the blood to flow into the aortic arch 63 and the innominate artery 64. Accordingly, the blood in the chamber 40 of the receptacle 4 may be effectively forced or pumped, by the diaphragm 43, to flow out of the receptacle 4 and to flow into the aortic arch 63 and the femoral artery 61 when the diaphragm 43 is expanded.

The blood pumping device includes no dead angles or dead portions. The blood in the chamber 40 of the receptacle 4 may be effectively forced or pumped out of the receptacle 4.

Accordingly, the blood pumping device in accordance with the present invention may be used to help draining the blood out of the left ventricle and for helping recovery of the heart or for facilitating the blood circulation of the patients.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. A blood pumping device comprising:
a receptacle including a chamber formed therein for receiving blood, and including a non-valved inlet opening for coupling to an aorta of a patient and for receiving blood from said aorta, and including a non-valved outlet opening for outward flowing of the blood, said receptacle including a surface having a peripheral portion and having a port provided therein,
a diaphragm received in said chamber of said receptacle, and including a space formed therein and including a peripheral portion coupled to said peripheral portion of said surface of said receptacle, and
means for pumping said diaphragm to draw the blood into said chamber of said receptacle and to pump the blood in said chamber of said receptacle out of said inlet opening and said outlet opening of said receptacle.

2. The blood pumping device according to claim 1, wherein said pumping means includes a pump coupled to said port of said receptacle for shrinking said diaphragm to draw the blood into said chamber of said receptacle, and for expanding said diaphragm to force and to pump the blood in said chamber of said receptacle out of said inlet opening and said outlet opening of said receptacle.
